(19) United States
(12) Patent Application Publication
(10) Pub. No.: US 2015/0027443 A1
(43) Pub. Date: Jan. 29, 2015

(54) LOW EMISSIVITY MATERIAL COATING OR LAYER

(71) Applicant: John Barr, Chesterfield, MO (US)

(72) Inventor: John Barr, Chesterfield, MO (US)

(21) Appl. No.: 13/949,753

(22) Filed: Jul. 24, 2013

(51) Int. Cl. A61M 16/08; A61M 16/00; A61M 16/06; A61M 16/16

(52) U.S. Cl. CPC A61M 16/0808; A61M 16/16; A61M 16/0875; A61M 16/0063; A61M 16/06
USPC 128/203.12

(57) ABSTRACT

A system for reducing condensation in CPAP devices including a CPAP device with air pump and humidifier, a tube connected in pneumatic communication with the air pump, a patient interface pneumatically connected to the hose, and a low-emissivity (below 0.3) outer coating covering the tube.

### TECHNICAL FIELD

The present novel technology relates to condensation management techniques for a gas flow delivery system, and more particularly, to a low emissivity CPAP tube.

### BACKGROUND

Several medical applications exist wherein a respiratory apparatus delivers a pressurized flow of breathable and humidified gas to a patient through a hose or tube. In particular, various forms of Positive Airway Pressure (PAP) devices, or continuous positive airway pressure machines (CPAP), are used to provide artificial ventilation to patients that experience sleep disordered breathing whereby a breathing machine pumps a controlled stream of air through a flexible tube. The flexible tube connects a filtered air pump to a mask worn over a patient's nose, mouth, or both. In addition, a typical PAP device utilizes a controlled air compressor to generate an airstream at constant pressure to hold the patient's airway open so uninterrupted breathing is maintained during sleep, and a humidifier adds moisture to the airstream being pumped to the user to avoid causing discomfort and nasal or upper airway dryness. Although humidifiers are an important addition, as they improve both patient compliance and patient comfort, the distance that humidified gas must traverse through a hose between the humidifier and the patient often results in condensation on the interior of the hose, which interrupts and negatively affects the machine's efficacy.

Condensation occurs when the warm, moist air from the humidifier cools before it can reach the warmth of the CPAP user's body, or their mask, while traversing between the hose and the machine. The amount of moisture that gas can hold is dependent upon the temperature of the gas; the higher the temperature, the greater the gas' capacity to hold water vapor. However, the temperature of the gas itself is also affected by ambient temperature. Temperature differences between the inside of the tubing and the external room temperature cause condensation to gather on the inside walls of the tubing. In the sense of temperature of a room, ambient temperature is influenced by a number of factors, including the weather outside, the quality of the insulation in the room, what or who is inside the room, humidity, and the use of heating and cooling systems. In the event the ambient temperature is lower than the temperature of the humidified gas, the gas/tube will lose heat to the surrounding air via convection and to surrounding objects with which it is in contact via conduction. The gas/tube will also lose heat to lower-temperature objects within unobstructed view, including through windows, via radiation.

Accumulation of water in CPAP tubing results in a disruptive gurgling noise and tube vibration, which adversely affects therapeutic pressure. Furthermore, condensation adds resistance to the CPAP tubing that can create large fluctuations in pressure at the mask. Condensed water can also be inadvertently inhaled. These irritations greatly reduce the continued use of the machine by patients.

In response to the condensation issue, which in extreme conditions can cause a genuine threat to life and health, several suggestions and solutions have been offered, all of which focus on reducing heat loss due to convection and conduction. For example, thin insulating “zipper jackets” made of generally synthetic material are commercially available for hoses, and electrically heated tubes have been developed to maintain a constant temperature at the mask and within the tubing regardless of varying ambient temperatures. Patients can manage the condensation characteristics of their CPAP machine by placing the device near the floor, insulating the hose as much as possible, and by adjusting the humidity level downward until the resulting condensation is tolerable; however, there is a seasonal effect on the amount of condensation that develops within the tubing, even when these factors are held constant. While techniques directed towards controlling heat loss via convection and conduction have been successful in reducing condensation in PAP hoses and tubes, they have not been able to fully eliminate the condensation problem. Thus, a need persists for a more effective technique for reducing or eliminating tube condensation. The present novel technology addresses this need.

### BRIEF DESCRIPTION OF THE DRAWINGS

FIG. 1 is a front perspective view of a CPAP machine according to a first embodiment of the present novel technology.

FIG. 2 is a front perspective view of a CPAP machine according to a second embodiment of the present novel technology.

### DESCRIPTION OF THE PREFERRED EMBODIMENTS

For the purposes of promoting an understanding of the principles of the novel technology and presenting its currently understood best mode of operation, reference will now be made to the embodiments illustrated in the drawings and specific language will be used to describe the same. It will nevertheless be understood that no limitation of the scope of the novel technology is thereby intended, with such alterations and further modifications in the illustrated device and such further applications of the principles of the novel technology as illustrated therein being contemplated as would normally occur to one skilled in the art to which the novel technology relates.

Contrary to intuition, which has led many to believe that maintaining the temperature in the hose and managing heat loss from convection and conduction through use of conventional insulation is the best available mechanism for reducing condensation in CPAP hoses, reducing heat loss resulting from radiation through a low-emissivity material is far more effective at reducing condensation. Radiation is energy transfer by emission of electromagnetic waves that carry energy away from the emitting object. The emissivity of a surface is the ratio of the radiation emitted by that surface to the radiation emitted by a perfect black body radiator at the same temperature. Materials with a high emissivity radiate a relatively large fraction of the theoretical maximum radiation at any given temperature. Likewise, materials with a high emissivity absorb a large fraction of radiation incident upon their surfaces. However, a low emissivity material reflects much of the radiant energy shining on it and likewise absorbs and radiates away only a small percentage of radiant energy.

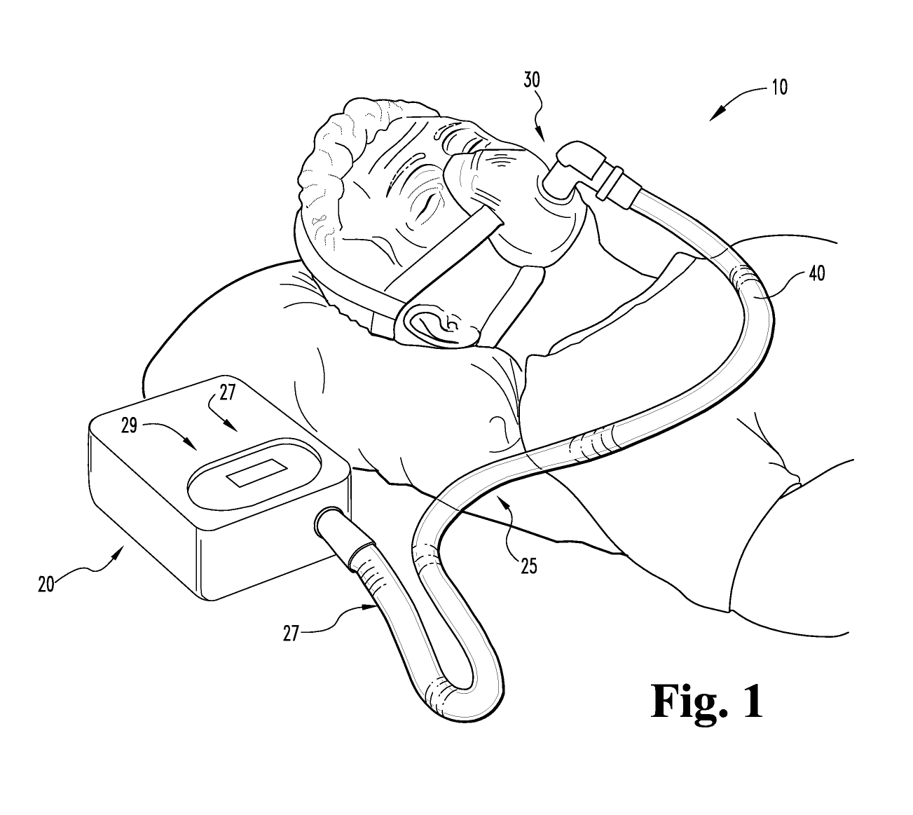

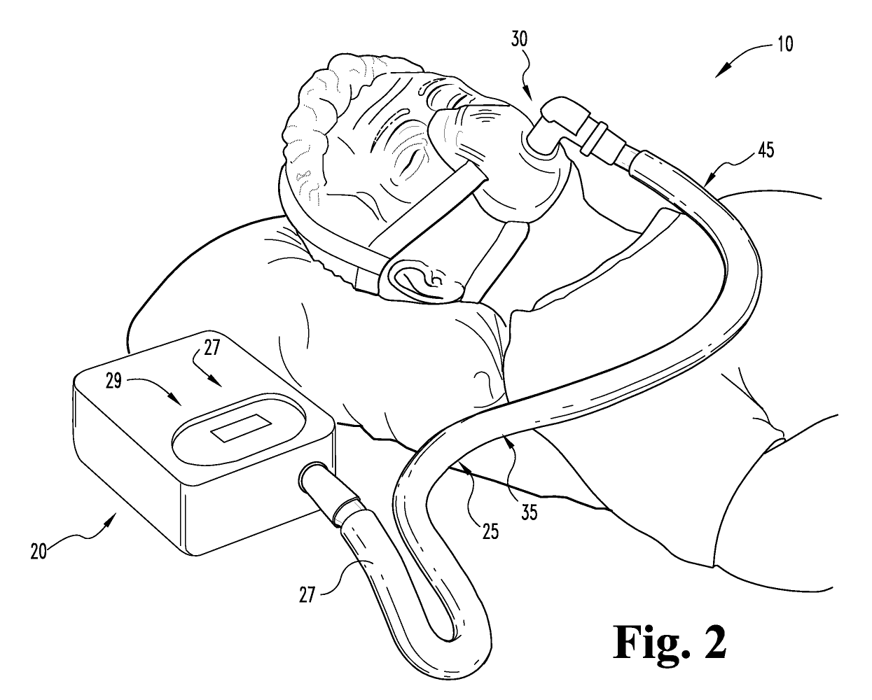

FIGS. 1-2 illustrate a first and second embodiment of the present novel technology, a system 10 for reducing condensation in Positive Airway Pressure devices, such as a CPAP device 20. One of ordinary skill in the art would understand that the present novel technology could also be used with a CAP device. Typically, a PAP or CPAP device 20 includes an air pump 27 and a humidifier 29 connected in fluidic communication with the pump 27. The system 10 generally includes a CPAP machine 20, which is responsible for generating airflow, a face mask 30, which can be any form of face enclosure that provides an entryway where air under positive pressure from the CPAP device 20 enters a patient's system, a hose 25 that connects the CPAP machine 20 to the patient's mask 30, and a low emissivity radiant barrier 35 generally encapsulating the hose 25.

Hose 25 typically defines a flexible thermoplastic polyurethane, or like material, connected in pneumatic communication to CPAP device 20 on one end and connected in pneumatic communication to face mask 30 on the other end. Flexible hosing 25 is typically on the order of about six feet in length, and is generally of sufficient width to provide steady airflow to a patient. In one embodiment, the CPAP hose 25 has a radiant barrier 35 defining an outer coating or layer of low-emissivity material that generally surrounds and protects the hose 25, reducing heat loss due to temperature differentials between the inside of the hose 25 and the ambient environment. The low-emissivity radiant barrier 35 typically has an emissivity below about 0.3; more typically below about 0.2; still more typically below about 0.1. The low-emissivity radiant barrier 35 may consist of a coating 40 that is applied directly onto the outer surface 27 of the hose 25 or it may be a protective sleeve 45 that encompasses the outer surface 27 of the hose 25. The radiant barrier 35 may also be integral to the tubing itself.

Compared to the current mechanisms for managing condensation in CPAP machines 20, which often add unwanted weight to the CPAP assembly, a low-emissivity radiant barrier 35 reduces heat transfer while adding only a small amount of weight to system 10. In addition, predicted condensation decreases when using an outer coating 40 or layer of a low-emissivity material, compared to the traditional measures of reducing heat loss by adding insulation and/or heating the tube. The rate of heat loss, or energy flow due to radiation from an object to its surroundings is governed by the Stephan-Boltzmann Equation:

P=eσA(T⁴−T_{c}⁴)

Wherein P=power (watts), ε=emissivity of an object, A=surface area of object (m²), σ=Stephan-Boltzmann constant (W/(m²K⁴), and T=temperature in Kelvin.

By way of example, and by using fairly conservative assumptions since outdoor temperatures can be lower and more hosing may be uncovered, an example of the rate of heat loss due to radiation follows. Assuming that 35 inches of a six foot hose is uncovered by pillows and blankets, and that one-third of the surface area of the exposed hose (circumference=3.5 inches) in radiant communication with an exterior wall or window, that the emissivity of the outer surface of the hose is about 0.8, and that given indoor and outdoor temperatures are 68° F. and 32° F., respectively, the Stephan-Boltzmann equation would predict a power loss of 2.15 Watts through the exposed hose via radiation. Over an eight hour period, such a power output would equate to approximately 62,000 Joules of energy removed from the hose. Using 40.69 kJ/mole as the heat of vaporization for water, 62,000 Joules would result in the condensation of 27 ml of water in excess of what would condense on a day where the outdoor temperature matched the indoor temperature of 68° F. Now, suppose the same set of geometric and temperature parameters as above, except now a coating or outer layer on the tube is applied that has an emissivity of 0.1 instead of 0.8. For the new lower emissivity and with boundary conditions remaining the same, the calculated condensation decreases from 27 ml to 3 ml.

While the novel technology has been illustrated and described in detail in the drawings and foregoing description, the same is to be considered as illustrative and not restrictive in character. It is understood that the embodiments have been shown and described in the foregoing specification in satisfaction of the best mode and enablement requirements. It is understood that one of ordinary skill in the art could readily make a nigh-infinite number of insubstantial changes and modifications to the above-described embodiments and that it would be impractical to attempt to describe all such embodiment variations in the present specification. Accordingly, it is understood that all changes and modifications that come within the spirit of the novel technology are desired to be protected.

What is claimed is:

1. A system for reducing condensation of humidified air in a Continuous Positive Airway Pressure (CPAP) gas flow delivery tube, comprising:
an elastomeric respiratory tube for delivering air flow from a Continuous Positive Airway Pressure (CPAP) system to a patient; and
a low-emissivity material generally covering the tube, wherein the low-emissivity material has an emissivity of less than about 0.3.

2. The system of claim 1 wherein the low-emissivity material is a protective sleeve at least partially enclosing the respiratory tube.

3. The system of claim 1 wherein the low-emissivity material is a coating adhered to at least a portion of the respiratory tube.

4. The system of claim 1 wherein the low-emissivity material is integral with the tube.

5. A system for managing condensation of humidified air in Positive Airway Pressure Devices, comprising:
a positive airway pressure device for generating an air flow;
a hose for transport of artificial ventilation in pneumatic communication with Positive Airway Pressure Device;
a patient interface pneumatically connected to the hose; and
an outer coating operationally connected to the hose;
wherein the outer coating has an emissivity less than about 0.3.

6. The system of claim 5 wherein the outer coating is a sleeve at least partially covering the hose.

7. The system of claim 5 wherein the outer coating is adhered to the hose.

8. The system of claim 5 wherein the outer coating has an emissivity less than about 0.2.

9. The system of claim 5 wherein the outer coating has an emissivity less than about 0.1.

10. A system for reducing condensation in Positive Airway Pressure Devices, comprising:
a positive airway pressure device having an air pump and humidifier;
a hose to transport the humidified air in pneumatic connection with the air pump;
a face mask in pneumatic communication with the hose; and
a low-emissivity barrier encapsulating the hose;
wherein low-emissivity barrier has an emissivity less than about 0.3.

11. The system of claim 10 wherein the low-emissivity barrier is integral with the hose.

12. A method for reducing condensation in the hose of a Continuous Positive Airway Pressure (CPAP) device, comprising:
a) connecting a Continuous Positive Airway Pressure (CPAP) machine in pneumatic communication with a patient via a tube;
b) flowing humidified air through the tube;
c) reducing radiative energy loss from the tube with a low-emissivity barrier positioned around the tube;
wherein low-emissivity barrier has an emissivity of less than about 0.3.

13. The method of claim 12 wherein the low-emissivity barrier is a sleeve at least partially covering the hose.

14. The method of claim 12 wherein the low-emissivity barrier is adhered to the hose.

15. The method of claim 12 wherein the low-emissivity barrier is integral with the hose.

16. The method of claim 12 wherein the low-emissivity barrier has an emissivity less than about 0.2.

17. The method of claim 12 wherein the low-emissivity barrier has an emissivity less than about 0.1.
